(19) United States
(12) Patent Application Publication
(10) Pub. No.: US 2021/0251794 A1
(43) Pub. Date: Aug. 19, 2021

(54) SHOULDERED ARM SLING WITH SHOULDER SADDLE, ETC.

(71) Applicants: Preston Nelson, Mount Vernon, IL (US); George A. Paletta, Chesterfield, MO (US)

(72) Inventors: Preston Nelson, Mount Vernon, IL (US); George A. Paletta, Chesterfield, MO (US)

(21) Appl. No.: 17/300,027

(22) Filed: Feb. 11, 2021

Related U.S. Application Data
(63) Continuation of application No. 62/995,878, filed on Feb. 17, 2020.

(51) Int. Cl. A61F 5/37

(52) U.S. Cl. CPC A61F 5/3738

(57) ABSTRACT

A combination of cushioning devices, as pillows, that have a surface area of one component of a hook-and-pile fastening device, and has applied to it a strap of the other hook-and-pile component, to allow the various pillow(s) to be applied to the limb of a patient during convalescence, or the pillows to be interconnected and stacked together, when used by the patient when resting, or sleeping, during such convalescence.

### FIELD OF THE INVENTION

The concept of this invention is to provide a pillowed support system to support a body part during rest and rehabilitation, and during convalescence, in order to provide comfort to the healing body part, support during said healing process, for the enhancing health and benefit of the patient. The pillowed support system includes a pillow, upon its outward surface having the application of one part of a hook-and-pile connecting system, while a strap like member carries the other of the hook-and-pile system, so that the one or more straps may be applied to the pillow around the limb or body part of the patient during the healing process.

### BACKGROUND OF THE INVENTION

Obviously, pillows and cushions have long been used to function as a comfortable support for the user, which in this instance, is to add to the comfort of the patient during a healing procedure.

Such pillows and cushions are widely used as an adjunct object for humans or other species, utilized for comfortably and ergonomically positioning about certain of the parts of the body for longer term positioning as during rest, and during sleep, as known and used for eons.

There have been specially designed orthopedic pillows for support to specific body parts, such as wedges, cervical pillows, and even extraordinarily large pillows which encompass the entire body, to add to comfort during the healing process. To the Applicants' knowledge, there have been no devices or system that has existed that allows one or more of smaller pillows capable of affixing to certain body parts to customizably adhere to the healing body part, such as an arm, or a leg, and other anatomy, which are recommended to remain immobilized following a surgical procedure, and during the long term of convalescence.

There are a variety of patents that relate to related technology, which can be seen in U.S. Pat. No. 6,957,462, that shows a pillow with a slidable strap through it, and clasp, for use for use for attachment to a portion of a user's body, and primarily used as a headrest. The published application to Moss, number U.S. 2013/0117939, shows a travel pillow for arm securement to rest against the head of the user. Another headrest.

Other publications show a pillow for application by straps or bandages to the foot of the user. Other publications show the use of medical bandages for holding a patient's broken arm against the body.

In addition. U.S. Pat. No. 6,874,182, shows a form of S-shaped pillow for flexible body support, along the length of the user, as previously referred to in this background.

### SUMMARY OF THE INVENTION

The broad concept of this invention is to provide the application of an orthopedic form of pillow like structures to certain body parts, during their healing process following surgery, or the corrections made to a broken bone, usually which entails many weeks of rest, to extend the convalescing process, during healing. For example, a broken arm may take six to eight weeks to recover back to a normal state after setting, and during that time, the patient must exercise significant caution in protecting the injured part, whether it be an arm, leg, ankle, foot, or the like, in order to achieve complete healing. In this particular instance, the more specific aspects of this invention include an especially structured pillow, one that is sized to conveniently fit contiguously with the injured body part, and then being held in position by means of cooperating straps, that may secure with the pillow by means of the hook-and-pile fastening means, otherwise known as Velcro, in order to add to the stability of the healing part, or to add to the rest and comfort during the convalescing. Obviously, other means for fastening may be utilized, such as the strap may be formed of plastic, and stitched in place upon the surface of the pillow may be resilient, to allow it to be expanded, for fitting of the specified body part, during its usage and application.

It is, therefore, the principal object of this invention to provide a customizable orthopedic support system, including pillows and straps, designed for use with specific body parts during a healing process, for the usage and comfort of the patient.

Another object of this invention is to provide for the secure fit of a customized pillow through the use of a variety of fastening means, such as especially designed straps, that adhere the pillow to the vulnerable appendage of the patient during convalescence.

Another object of this invention includes the application of a number of pillows, that may be secured together, also by hook-and-pile fastening means, and then held by specialized straps to the patient's limb or body part while undertaking a healing process, such as a broken leg, arm, back or hip surgery, or the like.

A further purpose of the invention is to provide for the patient an assurance of comfort and support, to provide the patient with peace of mind as during resting, or sleep, so that the patient need not worry about the fragile body part being displaced during such periods of convalescence, or during associated sleep, throughout the prolonged healing process.

Another object of this invention is to provide a pillow means that can aid in the recovery process for a patient following surgery, on a particular body part, to which the pillow is applied during usage.

These and other objects may become more apparent to those skilled in the art upon review of the Summary of the Invention provided herein, and upon undertaking a study of the Description of its Preferred Embodiments, in view of the Drawings.

### BRIEF DESCRIPTION OF THE DRAWINGS

In referring to the drawings:

FIG. 1 shows a pair of pillows, and various straps, which in this instance the straps are held to the pillow of the shown hook-and-pile fastening means;

FIG. 2 shows the comfort pillow applied to a broken arm, through the use of associated straps, even while the patient is asleep, to assure comfort and support for healing limb;

FIG. 3 shows the application of one or more pillows, by use of the associated straps, to a broken leg of the patient during the healing process; and

FIG. 4 shows a plurality of pillows connected together with hook-and-pile fastening means to apply along the length of the body portion of the patient, during convalescence, such as after back surgery.

### DESCRIPTION OF THE PREFERRED EMBODIMENTS

The concept of this invention, as previously summarized, is to provide an extended term cushioned support for the body, or an appendage, during rest and convalescence, as during sleep, and to prevent any unwanted impacting or displacement, or even shifting of the affected limb, during typically moments of rest or sleep. The object of the invention is to cushion the area that has been repaired, whether it be a broken limb, back surgery, surgery anywhere else on the body, as on the hip, where cushioning is desired to protect the healing area, to allow its sustained healing, without any disruption, as can be understood.

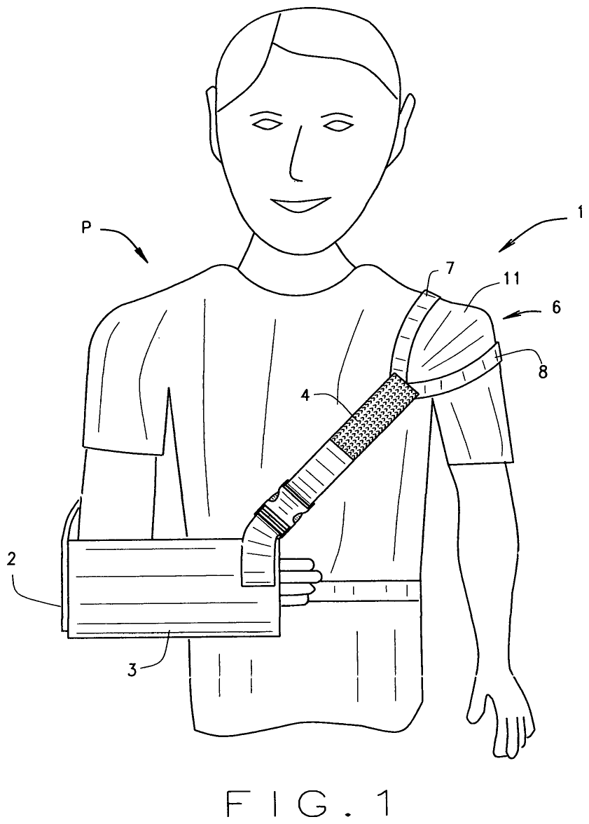

As can be seen in FIG. 1, there are disclosed a pair of pillows 1 and 2 and which has a segment of the hook-and-pile fastening means, such as Velcro, applied along select edges, or along an entire surface, as can be noted at 3 and 4. As show, these may be the soft pile portion of the fastener.

Obviously, other forms of fastening may also be considered, such as clasps, even potentially zippering, where a pair of pillows are desired to be interconnected together, to provide greater extensive cushioning for the affected limb, such as, for example, during the mending of a broken leg, that may require more surface cushioning to the patient to protect the limb during healing.

As also can be noted, various strapping, such as 5 and 6 may be provided, and these are furnished for either hooking the pillows to each other, in a number of customizable configurations, depending upon the number of pillows that may be employed within the system. These straps containing the hook portion of the Velcro at their ends, wrap over the body part to which the pillows are to be affixed, and then the straps are securely adhered to any selected portion of the pillow, which may be covered entirely with the pile-texture configuration, over all or part of its surfaces, as can be seen. The pillows may also be adhered to each other by the double sided Velcro tape, as noted at 6, in order to provide the extra support needed for the positioning and retention of the collected pillows at the precise location, where comfort and complete coverage is desired and required.

As stated, the pile portion, as at 3 and 4, for the pillows 1 and 2, may be along the peripheries, as noted in FIG. 1, or there may be such portions of the Velcro at various other locations upon the surface of each pillow, whether it be entirely around the perimeter, or even at select locations, as at a midpoint, or over the entire surface, to provide consistent connection of the pillow to the affected limb, by the strapping, to provide for the comfort of the patient, as during convalescence. Such is noted in said FIG. 1.

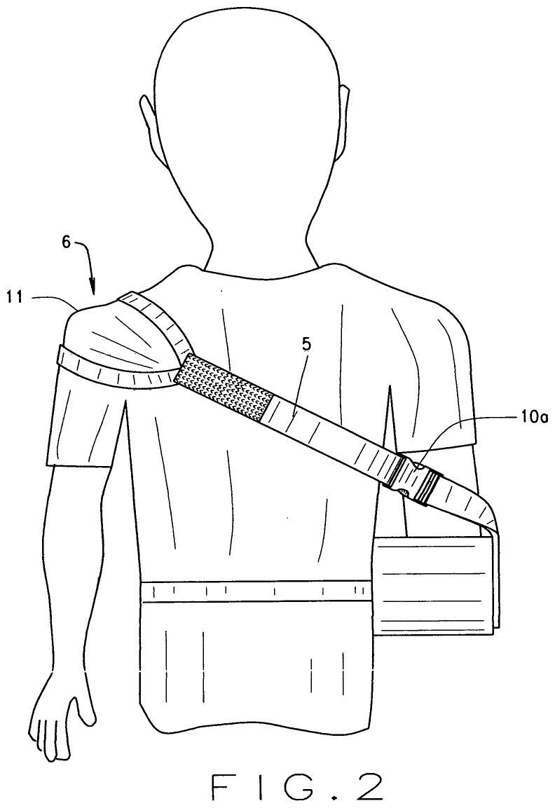

FIG. 2 provides an added view of the patient, one who is healing from either a broken arm, as the forearm, the bicep region, or an elbow, or is recovering from surgery at these locations, and discloses how the pillow 1 having a surface configuration of a part of the Velcro, as 3, applied to the surface of the shown pillow, and the straps, as at 5 and 6, may be wrapped around the arm, at the two shown locations, and applied to the surface of the pillow 1, in order to secure the pillow in place. As noted, the patient may be undertaking sleeping, at this time, or as during resting, and has the pillow both supporting the affected arm, and adding cushioning to the healing area, during such rest and sleep, as noted herein. Obviously, such a cushioning means, through application of the pillow, may be applied even during the working hours, just to add comfort to the patient, and the affected arm, as can be understood.

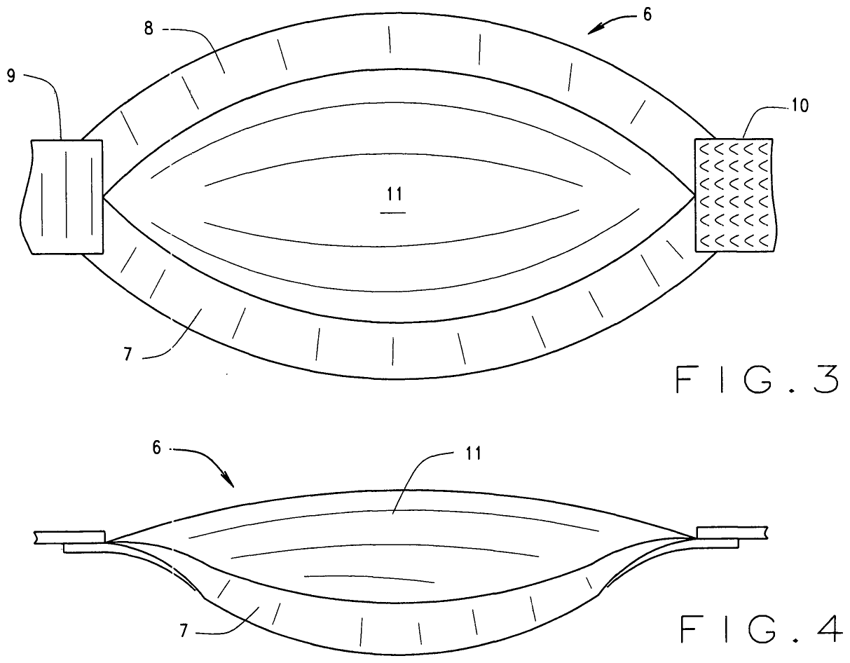

FIG. 3 provides a similar type of view to that of FIG. 2, and discloses how a pair of the pillows, 1 and 2, may be interconnected to each other by means of a Velcro strap, such as shown at 6, and then the pillows may be held by the strapping 5, through the application of its hook-and-pile areas 3 and 4, as draped around the affected leg of the patient, at multiple locations, as can be noted. This combination, through the use of this system, provides cushioning throughout the entire length of the leg, as may be required for the extent of the injury that has been sustained, over the entire length of the leg, and which requires protection, and cushioning, during healing.

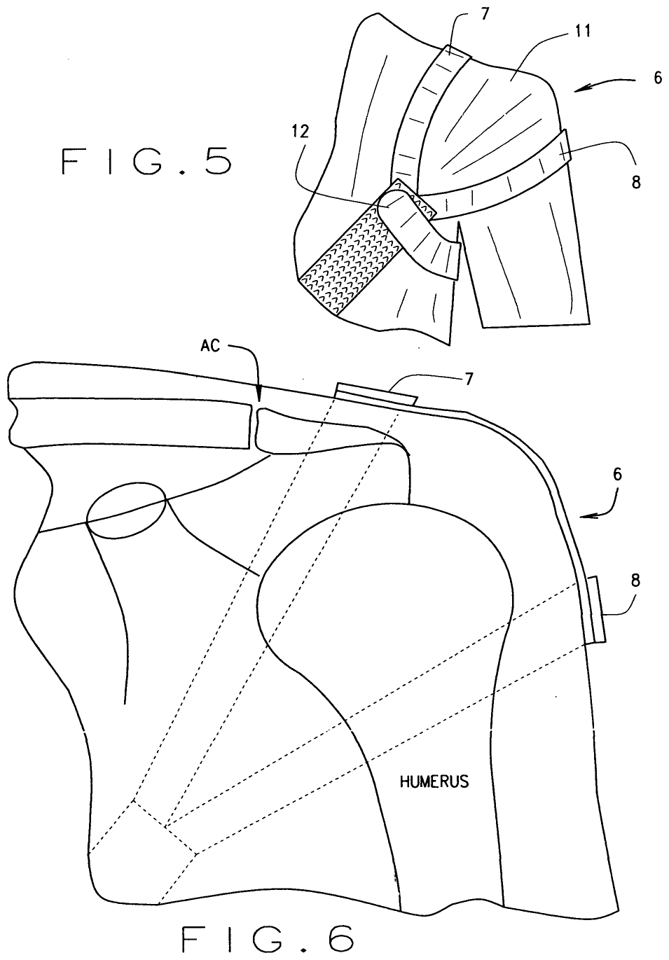

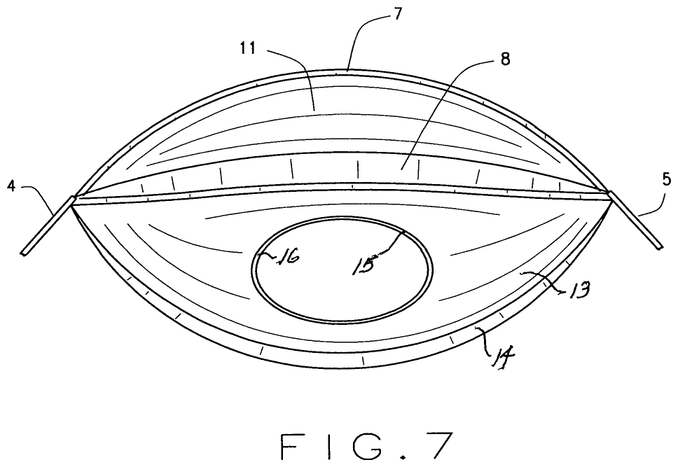

FIGS. 4, and 4(a) and 4(b), show how the various pillows, as noted at 7, may be interlinked together, in order to build up the surface area of the combination of the cushioning pillows, when said substantial cushioning is required, to add to the comfort of the shown patient. As can be noted, the various pillows 7 may have a surface texture of a very short type of loop or pile portion of the fastening means, known as Velcro, and the desired soft surface texture of the shown pillow (pillows) may be applied to one or both surfaces, as at 8, of each of the identified pillows. Then, a double sided form of the other hook or loop type member of the Velcro, as noted at 6, may be applied between the various shown pillows, in order to adhere the same together, to build up a nesting of these assembled pillows, to the extent that patient requires, in order to attain the type of cushioning, and comfort, as desired, during convalescence. For example, the combination of three pillows as noted in FIG. 4(a) may be applied under the knees of the patient, as shown at 9, to add to the comfort at that location. And combination of pillows, as shown in FIG. 4(b), may be applied under the upper torso and head area of the patient, as noted at 10, to add to the comfort, and support of the patient, during such convalescence, as for example, after back surgery.

These are examples as to how the various combination of the pillows, that may have a partial or full surface area of one component of the hook-and-pile fastening means, while the various strapping may have the other of the loop, hook, or pile portion of Velcro, in order to secure to the pillows, and adhere them around the affected and repairing areas of the body, or the pillows may be looped together, as noted in FIG. 4, and held by the double sided strapping, as at 6, for location under various portions of the patient's body, to add to the support and comfort of the patient, during the healing process.

Variations or modifications of the subject matter of this invention may occur to those skilled in the art upon review of the disclosure as, provided herein. Such variations, if within the spirit of this invention, are intended to be encompassed within the scope of any claims to patent protection issuing upon this development. The various descriptions of the components of the invention, its depiction in the drawings, and detailed explanation in the Specification, are primarily set forth for illustrative purposes only.

We claim:

1. A cushioning device, such as a pillow, having one segment of a hook-and-pile fastening device connected to a portion or all of its exposed surfaces, and a strap having the other of the hook-and-pile fastening device secured thereto, so that the strap may be applied around the limb of a convalescing patient and securing with the surface of the pillow to hold said pillow contiguously on the limb of the patient during healing.

2. The cushioning device of claim 1 wherein the portion of the hook-and-pile fastening device can be applied over the entire one side surface of the pillow, to add to the convenience of the application of the strap at various locations to secure the pillow upon the limb of the affected patient.

3. The cushioning device of claim 2, where in said portion of the hook and pile fastening means is applied to both surfaces of the said pillow.

4. The cushioning device of claim 1, where there are a plurality of pillows, each having one portion of the hook-and-pile fastening means secured to a part of its surface, and a strap having the other of the hook-and-pile fastening means secured to both of its surfaces in order to secure a plurality of pillows together in preparation for usage and support of the patient during convalescence.

5. The cushioning device of claim 1, wherein there are a plurality of pillows, and said straps provided for securement of the plurality of pillows together for application in support of a patient during convalescence.
